(19) United States
(12) Patent Application Publication
(10) Pub. No.: US 2015/0150655 A1
(43) Pub. Date: Jun. 4, 2015

(54) CAD / CAM BASED METHOD FOR THREE-DIMENSIONAL QUALITY CONTROL OF INSERTED IMPLANTS OR RESTORATIONS BY CORRELATING PREOPERATIVE RADIOGRAPHS WITH POSTOPERATIVE OPTICAL SCANS AND TO AVOID POSTOPERATIVE RADIOGRAPHS

(71) Applicants: Elmar Frank, Besigheim (DE); Sigrid Frank, Besigheim (DE)

(72) Inventors: Elmar Frank, Besigheim (DE); Sigrid Frank, Besigheim (DE)

(73) Assignee: SIRONA DENTAL SYSTEMS GMBH, Bensheim (DE)

(21) Appl. No.: 14/550,023

(22) Filed: Nov. 21, 2014

(30) Foreign Application Priority Data
Dec. 3, 2013 (DE) 10 2013 224 778.8

(51) Int. Cl. A61C 8/00; A61B 6/14; A61B 6/00; A61C 9/00

(52) U.S. Cl. CPC A61C 8/0089; A61C 9/0053; A61B 6/14; A61B 6/5247

(57) ABSTRACT

A method for determining a quality feature of a dental treatment comprises the detection of a patient's radiographic image data set before the creation of a drill hole for a dental implant, the detection of a patient's non-radiographic image data set after the creation of the drill hole for a dental implant by means of a drilling tool, the correlation of the radiographic image data set and the intraoral non-radiographic image data set, and the determination of the quality feature from the correlation of the radiographic image data set and the non-radiographic image data set. A device (1) that comprises a computer (10), an interface (11) for detection of a radiographic image data set, and an interface (12) for detection of a non-radiographic image data set is equipped to carry out this method. This detection of the quality feature can take place during implantation, after it is concluded, or after mounting of a dental treatment on the implant(s) is concluded.

### TECHNICAL FIELD

The present invention relates to a method for determining a quality feature of a dental treatment. In addition, the present invention relates to a computer program that carries out all the steps of the inventive method when it is running on a computer, as well as to a data medium that stores this computer program. Finally, the invention relates to a device designed for carrying out the inventive method.

### PRIOR ART

After successful implantation and/or incorporation of certain forms of dentures, the treating doctor must check whether he has achieved the planned implant position in the jaw and/or whether the denture has been positioned without any gaps. An X-ray is traditionally taken for this purpose. This is usually done as a two-dimensional X-ray for reasons of cost and for protection against radiation, because 3D X-rays involve a higher radiation dose and are more expensive. A check is thus performed at least in the vertical direction, documenting that the implant that has been inserted is properly positioned and does not contact any endangered neighboring structures such as neighboring dental roots, nerves, hollow spaces, etc. The third dimension perpendicular to the plane of the image cannot be checked with a 2D view, because on the one hand the X-ray is taken in two dimensions, and on the other hand even if it were taken in three dimensions, it might not supply the desired information with the required fineness due to artifacts, because digital volume tomography (DVT) would create problematic artifacts in the tomographic data in the immediate vicinity of metal structures. Thus at least two X-rays are usually taken in the case of an implant treatment, one preoperatively and one postoperatively. The patient is therefore exposed to an elevated radiation dose in comparison with a single X-ray.

Furthermore, a follow-up X-ray is also created in many cases after mounting of the implant treatment, such as an abutment, a bar or a screw-down crown on an implant, to check whether the treatment is seated correctly on the implant, i.e. for example whether it forms a “tight seal” and has been screwed in and/or mounted up to a stop. This is done in particular when the gap to be investigated is subgingival, so that it is not detected by a mechanical palpation examination using a dental probe. Since the final restoration is not usually mounted immediately after implantation, i.e., is not included in the X-ray for checking whether the correct implant position has been achieved, an additional (third) X-ray is even taken here if needed.

There is not yet a known method that uses only the initial X-ray needed for planning and avoids additional X-rays by combining preoperative X-ray data and radiation-free postoperative surface scan data.

DE 199 52 962 B4 describes how surface scan data and X-ray data are combined to derive drilling templates for implants.

WO 2013/045462 A1 describes how the mounting of a drilling template on the teeth before an operation can be correlated by an optical scan, as well as how the drilling direction can be converted back to a correlated radiographic image, if the extrapolated implant position is known from additional information.

However, neither of these prior art documents describes how to avoid the additional X-rays that are still customary today in the treatment process after the initial diagnostic and/or planning X-ray. Furthermore, checking on the seating of a drilling template is no guarantee that an implant is in fact seated in the position predetermined by the drilling template. Misalignment of the implant may occur, for example, if the drilling template is used only for a pilot bore, but not for a subsequent reaming bore.

### DISCLOSURE OF THE INVENTION

The inventive method for determining a quality feature of a dental treatment includes detection of a patient's radiographic image data set, in particular intraoral, before creation of a drill hole for a dental implant, detection of a patient's non-radiographic, in particular intraoral, in particular optical, image data set after the creation of the drill hole for a dental implant by means of a drilling tool, the correlation of the radiographic image data set and of the non-radiographic image data set, and determination of the quality feature from the correlation of the radiographic image data set and the non-radiographic image data set. It may be used independently of whether or not a drilling template is used to create the drill hole. If a drilling template is used, it permits a result-oriented 3D quality control of the drilling template.

In one embodiment of the invention, the quality feature is the location of the drill hole, i.e., its size and position, during or after performing the drilling. In this way, the drilling progress can be checked intraoperatively. By checking the drilling depth and the angulation of the drilling, insertion of an implant into an improperly drilled hole can be prevented. In particular when setting several implants in one session, implant planning can be updated intraoperatively and adjusted if necessary. Before the detection of the non-radiographic image data set, in particular a measuring body and/or a scan body can be inserted into the drill hole. This may be done, for example, by attaching it to the end of a drill, instead of to the end of an insertion tool and/or of a handpiece. Alternatively, it is also possible for the drilling tool to be left in the drill hole during the detection of the non-radiographic image data set. Its top side can then be detected by a camera and thus recorded in the non-radiographic image data set. This permits, for example, a check of a root treatment hole. An endodontic drill and/or root canal drill is then detected from above non-radiographically, in particular optically. Its depth and position in the dental root can thus be visualized by converting back, via the surface data, to volume data of the radiographic image data set without requiring that a new X-ray be taken. According to the prior art, up to now a new X-ray showing the endodontic drill in the root canal would typically be created for this purpose, leading to another X-ray and thus to an increase in the radiation burden for the patient. It is also possible to insert a final root filling post into the drill hole before the detection of the non-radiographic image data set, wherein its position may represent the quality feature, because it has a predefined length. An X-ray master point image and/or a follow-up image that would otherwise be stipulated could be omitted in this way.

In another embodiment of the inventive method, an implant is inserted into the drill hole by means of an insertion tool before the detection of the non-radiographic image data set. The quality feature is preferably the position achieved by the implant during or after its insertion by implantation. In the case of an implant with a visible cervical end, it is possible to detect directly the implant and/or its upper surface parts that are visible in an optical surface scan and to record them in the non-radiographic image data set. Before the detection of the non-radiographic image data set, however, a measuring body in particular may also be inserted into the implant. This can be detected in the non-radiographic image data set. In addition, it is possible in particular to leave the insertion tool in the implant during the detection of the non-radiographic image data set. Many implant systems are supplied with a handle that is screwed to the implant and serves to remove the implant from its packaging in a sterile manner and to introduce it into the drill hole. This handle can be recognized in the detected non-radiographic image data set. The position of the implant can be determined and imaged by means of the inventive method without necessitating an additional X-ray after insertion of the implant. Deviations that occur during implantation can be checked by comparing the actual position of the implant with its planned position, by displaying both on one display at the same time, for example.

In another embodiment of the invention, an implant treatment is connected to the implant before the detection of the non-radiographic image data set. The quality feature here is the position of the implant treatment with respect to the patient's remaining teeth. The implant treatment may be, for example, an abutment, a bar, a screw-down crown or a bridge. This can be attached to the implant by mounting or screwing. If the implant treatment was created by means of a CAD/CAM system, there is already a digital data set of its surface. Alternatively, the restoration shape can also be detected digitally at first in the inventive method, by taking an optical surface scan or an X-ray of the implant treatment outside the body of the patient. Then the visible components of the implant treatment can be determined from the non-radiographic optical image data set. For this purpose it is advantageous to optically scan the implant treatment directly intraorally to prevent casting errors. Alternatively, however, it is also possible to first perform a traditional casting of the implant treatment and to transfer the casting to a model, which is then scanned optically. The position of the implant treatment with respect to the remaining teeth as a quality feature makes it possible to ascertain whether the implant treatment is mounted tightly and correctly on the implant and/or how it is positioned in the X-ray volume, by determining it, i.e. its position in the optical scan, by means of surface fitting algorithms. Since the position of the non-radiographic optical data with respect to the X-ray data is known, the position of the implant treatment in the X-ray data is also known.

It is preferable that the radiographic image data set is a three-dimensional radiographic image data set and that the non-radiographic image data set is a three-dimensional optical surface scan image data set. The radiographic image data set originates in particular from panorama layer images, tomosynthetic images or computer tomographic images. Of these, digital volume tomographs (DVT) are particularly preferred. Markers may be used for correlation of the radiographic image data set and the optical image data set. If these markers are visible in an X-ray as well as in an optical image of the intraoral cavity, then a correlation of the radiographic image data set and the optical image data set can be achieved by making the markers coincide. A correlation of the image data sets can also be obtained by converting measured data sets of a three-dimensional optical image into pseudo-X-rays, with the assumption of standard X-ray adsorption values. The actual X-ray and the pseudo-X-ray can be made to coincide from several directions, for example on the basis of longitudinal and transverse sections in a panorama X-ray. A correlation may also occur by extracting surface shapes, as detected in an optical image, at least partially from the radiographic image data, and then making these shapes coincide with the optical image data set. This can be done automatically as well as interactively. The correlation permits model diagnostics, because the position can be checked for whether it is prosthetically correct and at the same time used for production of a dental prosthesis (to be supplied immediately).

It is preferable that there take place a quality assessment of the dental treatment by comparing the quality feature with a default value from a CAD/CAM system.

The inventive computer program performs all the steps of the inventive method when it is run on a computer. The inventive data medium stores the inventive computer program.

A device that is equipped to carry out the inventive method comprises a computer, an interface for detection or input of a radiographic image data set, and an interface for detection or input of a non-radiographic image data set.

### BRIEF DESCRIPTION OF THE DRAWINGS

Exemplary embodiments of the invention are illustrated in the drawings and explained in greater detail in the following description.

FIG. 1 shows a device for determining a quality feature of a dental treatment according to one embodiment of the invention.

FIG. 2 shows a schematic sectional diagram of a jaw detail of a patient in a method according to one embodiment of the invention.

FIG. 3 shows a schematic sectional diagram of a jaw detail of a patient in another embodiment of a method according to the invention.

FIG. 4 shows a schematic diagram of a jaw detail of a patient in yet another embodiment of the inventive method.

FIG. 5 shows a schematic sectional diagram of a jaw detail of a patient in yet another embodiment of the invention.

FIG. 6 shows a schematic sectional diagram of a jaw detail of a patient in yet another embodiment of the inventive method.

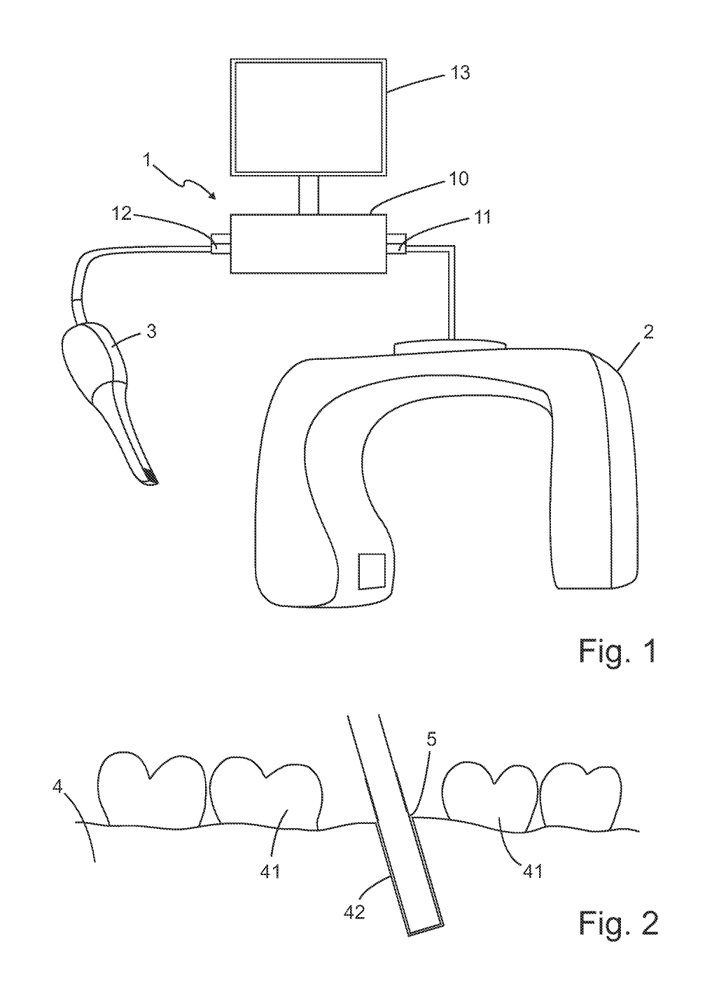

### EXEMPLARY EMBODIMENTS

In the exemplary embodiments of the inventive method as described below, a device 1 having a computer 10 with two interfaces 11, 12 is used. The first interface 11 is connected to a three-dimensional digital volume tomography system 2 (GALILEOS by the present applicant). The second interface 12 is connected to an intraoral three-dimensional optical camera 3. The computer 10 is also connected to a monitor 13, on which the results of a correlation between a radiographic image data set received via the first interface 11 and an optical image data set received via the second interface 12 can be displayed. In all the embodiments of the inventive method described below, an intraoral radiographic image data set of a patient is created first by means of the digital volume tomography system 2 before it is subjected to the additional treatment described below.

In a first embodiment of the inventive method, a drill hole 42 is created in a jaw 4 of a patient, who has the dentition 41, by means of a drilling tool 5. The drilling tool 5 is left in the drill hole 42. Next, an intraoral optical image data set of the patient is created by means of the intraoral camera 3. This is forwarded via the second interface 12 to the computer 10, where the correlation with the radiographic image data set is determined. From the correlation, the position of the drill hole 42 as a quality feature of the drilling operation is determined and displayed on the display 13.

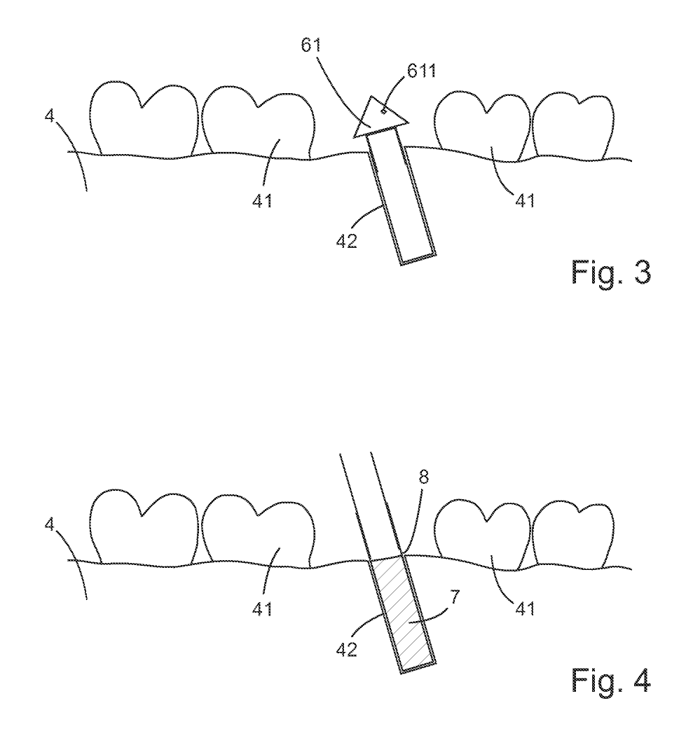

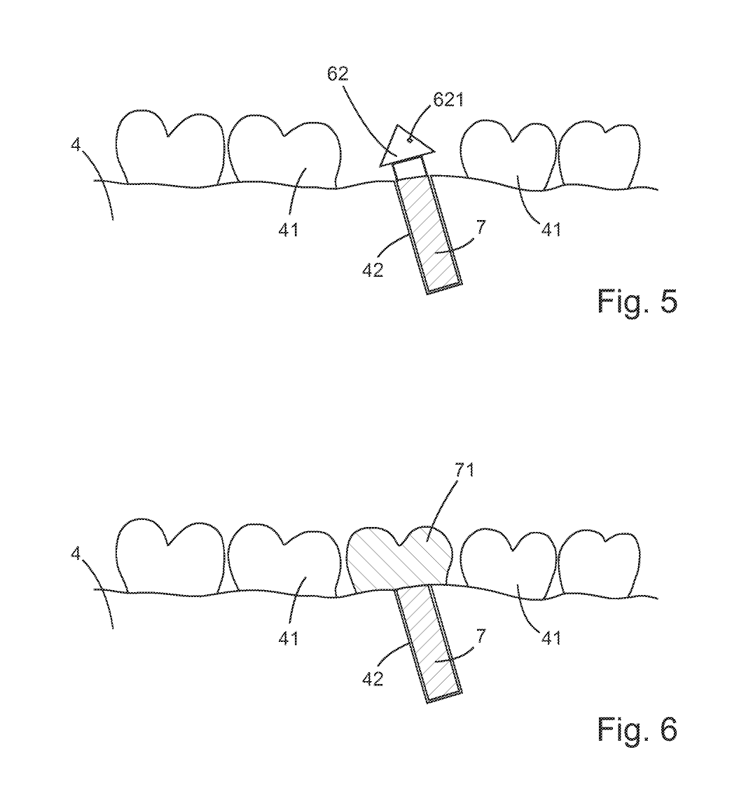

In a second embodiment of the inventive method, which is shown in FIG. 3, the drilling tool 5 is removed from the drill hole 42 and a measuring body 61 is inserted into the drill hole 42 instead. The measuring body 61 has an optical marking 611 on its surface. Then the patient's intraoral cavity is scanned optically by the intraoral 3D camera 3, and the produced optical image data set is forwarded to the computer for correlation with the radiographic image data set. The measuring body 61 is detected here on the basis of the marking 611. The correlation result is displayed on the display 13, as in the first embodiment of the inventive method.

In a third embodiment of the inventive method, which is shown in FIG. 4, the inventive method is used to determine the position of an implant 7 in the jaw 4. The implant 7 is inserted into the drill hole 42 by means of an insertion tool 8. The insertion tool 8 is then left on the implant 7 and next an intraoral scan is performed by means of the three-dimensional intraoral camera 3. The computer 10 performs a correlation of the resulting optical image data set with the radiographic data set, the position of the implant being determined from the position of the insertion tool 8 that is detectable in the optical image data set. The correlation result is displayed on a display 13.

In a fourth exemplary embodiment of the invention, the insertion tool is removed from the implant 7 and a measuring body 62 is instead attached to the implant 7. The measuring body 62 has an optical marking 621. Next, as in the second embodiment of the inventive method, an intraoral scan of the patient is performed, generating an optical image data set in which the measuring body 62 can be identified on the basis of its marking 621. The position of the implant 7 can be determined from the correlation of the optical image data set with the radiographic image data set and displayed on the display 13.

FIG. 6 shows a fifth embodiment of the inventive method. An abutment is screwed onto the implant 7 as the implant treatment. A crown 71 that is manufactured by means of a CAD/CAM system (CEREC system by the present applicant) is cemented onto the abutment. An intraoral scan is performed by means of the intraoral 3D camera 3. When the resulting optical image data set is correlated with the radiographic image data set, the position of the crown 71 in relation to the remaining teeth 41 can be displayed on the display 13 and a check can be performed by comparison with the data of the CAD/CAM system to ascertain whether the crown 71 is mounted tightly and correctly on the implant 7.

By using the embodiments of the inventive method described above, it is possible to avoid the use of X-rays, which are standard today, after the creation of the drill hole 42, after insertion of the implant 7 and after attachment of the crown 71. This reduces the radiation burden for the patient, which leads to a positive effect, from the standpoint of protection against radiation, in postoperative diagnostics. Furthermore, the optical measurement is more accurate than follow-up by means of an additional X-ray. This is due to the fact that both the resolution and the precision of an optical measurement method exceed those of a radiographic measurement method. Furthermore, a traditional comparison of a plurality of X-rays does not take place automatically but instead is performed visually by the doctor. The inventive correlation of the radiographic image data set and the optical image data set makes it possible to specify and visualize deviations in the implant position or the position of an abutment in micrometers or degrees of inclination.

1. A method for determining a quality feature of a dental treatment, comprising:
detection of a patient's intraoral radiographic image data set before the creation of a drill hole for a dental implant,
detection of a patient's non-radiographic image data set after the creation of the drill hole for a dental implant by means of a drilling tool,
correlation of the radiographic image data set and the non-radiographic image data set, and
determining the quality feature from the correlation of the radiographic image data set and the non-radiographic image data set.

2. The method according to claim 1, characterized in that the quality feature is the position of the drill hole during the drilling or after the drilling has been performed.

3. The method according to claim 2, characterized in that during the detection of the non-radiographic image data set, the drilling tool is left in the drill hole.

4. The method according to claim 2, characterized in that before the detection of the non-radiographic image data set, a measuring body is inserted into the drill hole.

5. The method according to claim 2, characterized in that before the detection of the non-radiographic image data set, a root filling post is inserted into the drill hole.

6. The method according to claim 1, characterized in that before the detection of the non-radiographic image data set, an implant is inserted into the drill hole by means of an insertion tool.

7. The method according to claim 6, characterized in that the quality feature is the position reached by the implant 0 during or after the insertion by implantation.

8. The method according to claim 7, characterized in that the insertion tool is left in the implant during the detection of the non-radiographic image data set.

9. The method according to claim 7, characterized in that before the detection of the non-radiographic image data set, a measuring body is introduced into the implant.

10. The method according to claim 6, characterized in that before the detection of the non-radiographic image data set an implant treatment is connected to the implant, and that the quality feature is the position of the implant treatment with respect to the patient's remaining teeth.

11. The method according to claim 1, characterized in that the radiographic image data set is a three-dimensional radiographic image data set, and the non-radiographic image data set is a three-dimensional optical surface scan image data set.

12. The method according to claim 1, characterized in that a quality assessment of the dental treatment takes place by comparing the quality feature with a default value from a CAD/CAM system.

13. A computer program that carries out all the steps of a method according to claim 1 when it is run on a computer.

14. A data medium, characterized in that it stores a computer according to claim 13.

15. A device, comprising a computer, an interface for detection or input of a radiographic image data set, and an interface for detection or input of a non-radiographic image data set, characterized in that it is equipped to carry out a method according to claim 1.
